(19) United States
(12) Patent Application Publication
(10) Pub. No.: US 2017/0119471 A1
(43) Pub. Date: May 4, 2017

(54) Augmented Reality Imaging System for Cosmetic Surgical Procedures

(71) Applicant: Illusio, Inc., San Clemente, CA (US)

(72) Inventors: Ethan WINNER, San Clemente, CA (US); Preston PLATT, Walnut Creek, CA (US)

(73) Assignee: Illusio, Inc., San Clemente, CA (US)

(21) Appl. No.: 15/188,776

(22) Filed: Jun. 21, 2016

Related U.S. Application Data
(60) Provisional application No. 62/250,888, filed on Nov. 4, 2015.

(51) Int. Cl. A61B 34/10; G06F 19/00; G06T 19/00; A61B 34/00

(52) U.S. Cl. CPC A61B 34/10; A61B 34/25; G06F 19/3437; G06T 19/006; A61F 2/12

(57) ABSTRACT

An augmented reality imaging system for cosmetic surgical procedures. In a breast augmentation procedure, a virtual breast image is generated and overlaid on a target marker covering a patient's real breasts such that the patient can see her real body with virtual breasts at the location of her real breasts. The patient views this augmented reality image on a mobile device such as a tablet computer. By use of low polygon graphics and a set of artistic sliders on the mobile device, a surgeon can manipulate and deform the virtual breasts in real time to produce lifelike outcomes of various surgical options.

### CROSS-REFERENCE TO RELATED APPLICATIONS

This application claims the benefit of priority under 35 USC 119 of provisional application No. 62/250,888, filed in the United States on Nov. 4, 2015.

### BACKGROUND OF THE INVENTION

Most patients considering a cosmetic surgical procedure, such as breast augmentation, experience some level of “size and outcome” anxiety. The most common concern for cosmetic surgery patients is what they will look like after the surgery. This is an issue for both surgeons and patients. There are approximately 8,000 plastic surgeons in the United States who perform approximately 1.8 million surgical procedures per year. Often, the patient may have unrealistic expectations, or expectations that differ from the expectations of the surgeon. With respect to breast augmentation, for example, 20% of breast augmentation surgeries result in re-operation, and 20% of those re-operations are due to the patient's dissatisfaction with the size or style resulting from the breast augmentation.

To date, there has not been an effective tool to provide a real-time visual representation of what a patient can expect from a cosmetic surgical procedure such as breast augmentation or implant surgery. Conventional approaches have included before and after pictures of other patients, manipulated or cropped photographs that make it difficult to evaluate how the implants will actually appear, or the use of cumbersome and expensive equipment to create a manipulated three-dimensional rendering of a torso. The resulting image from such three-dimensional rendering resides on a computer screen and the patient sees only an animated representation of their torso. The image is not fluid, is not on their person, and does not include an image of their face. Moreover, conventional approaches use algorithms based on a photograph of the patient and measurements of the proposed implants in order to calculate the possible outcome of surgery.

### SUMMARY OF THE INVENTION

The present invention addresses these shortcomings of conventional technology, and via use of augmented reality (AR) technology, places a virtual image on the real patient to allow the patient to preview the expected results of surgery using a mobile device, such as a tablet computer, as a virtual mirror. Complete confidence can be instilled in the patient by enabling them to view an extremely accurate preview of their expected post-operation appearance that resembles looking at themselves in a full length mirror, and in the comfort of their own home. The present invention answers the question of “What will I look like?” and allows the patient to see their future self.

The present invention provides a powerful new tool for aligning patient expectations with actual achievable outcomes. By this tool, patients have the ability to consider their options, evaluate different outcomes, and collaborate with the surgeon until both are aligned on objectives and results. Advantages are provided for both surgeons and patients. From the perspective of both surgeons and patients, the surgeon/patient communication gap is closed, both surgeon and patient see the same preview of expected post-operation appearance and thus have aligned expectations, and patient satisfaction is increased. From the perspective of the patient, decision making is easier, anxiety is reduced, confidence in achieving expected results is increased, the need for re-operation or revision procedures is reduced, and the ability to have a home experience in previewing the expected post-operation appearance increases comfort. From the perspective of the surgeon, there is the ability to increase patient conversion and reap the ongoing rewards of positive patient satisfaction, referrals and reviews.

The patient is able to interact with a virtual image on her person and gain comfort with their expected post-operation appearance. In particular, the patient can view themselves in real time with virtual breasts overlaid on their person. By use of augmented reality (AR), a virtual image is placed on a real patient to allow them to view themselves using a mobile device as a virtual mirror. The mobile device may be, for example, a tablet computer such as an iPad® by Apple. With respect to breast augmentation, the patient can see what her new breasts will look like with a complete view of her entire body. The surgeon is able to manipulate the virtual breasts to show various achievable results. The patient is thereby able to see different size options, and see the virtual breasts move with her as she moves. The patient is able to turn shoulder-to-shoulder and have close-up as well as a wide angle view of herself with the image staying on her person, and in proper perspective with regards to size and three-dimensional viewing, as she moves.

Conventional systems that make calculations based on photographs of the patients and measurements of the proposed implants do not provide the most accurate post-surgical representation based on the unique characteristics of each patient. Variables such as age, skin elasticity, firmness of breast tissue and placement of the implant are difficult to capture. The present invention provides a far more accurate representation by use of a virtual breast model using low-polygon graphics. Through use of a set of artistic sliders, the surgeon can easily produce lifelike outcomes of every possibility. In addition, the present invention can be used as a teaching tool to demonstrate bath good and bad outcomes of different options.

### BRIEF DESCRIPTION OF THE DRAWINGS

FIG. 1 is a diagram of an initial breast mesh according to the present invention.

FIG. 2 is a diagram of an initial nipple mesh according to the present invention.

FIG. 3 is a diagram of a base joint and curve rig according to the present invention.

FIG. 4 is another diagram of a base joint and curve rig according to the present invention.

FIG. 5 is a diagram illustrating various breast deformations achieved using the base joint and curve rig of the present invention.

FIG. 6 is a diagram of a user interface and display of a mobile device showing sliders for virtual breast deformation according to the present invention.

FIG. 7A is a diagram illustrating a tracking marker on a patient according to the present invention.

FIG. 7B is a diagram showing the patient with the tracking marker of FIG. 7A viewing an augmented reality image on a mobile device.

FIG. 7C is a diagram showing the augmented reality image that is displayed on the mobile device of FIG. 7B.

FIG. 8 is a block diagram of an exemplary mobile device on which the mobile application of the present invention is carried out.

### DETAILED DESCRIPTION OF THE INVENTION

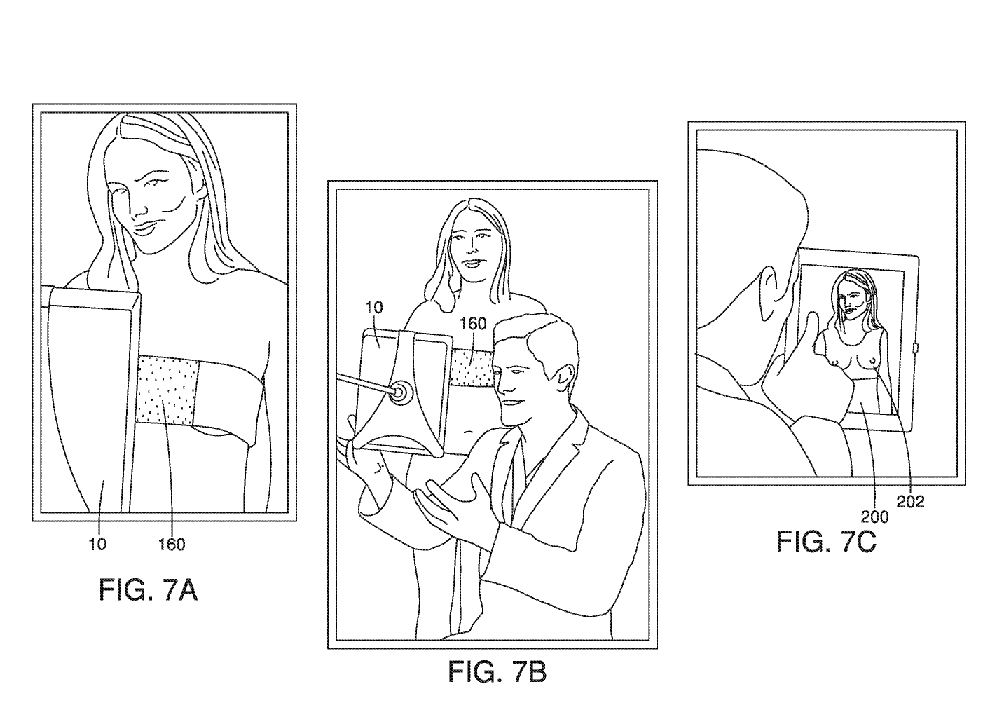

The present invention uses augmented reality (AR) to generate a virtual image of breasts that is overlaid on a tracking marker 160, such that a patient can view herself with the virtual breasts 202 on her person 200 (see FIGS. 7A-7C). Augmented reality (AR) is a live, direct or indirect, view of a physical, real-world environment whose elements are augmented (or supplemented) by computer-generated sensory input such as sound, video or graphics.

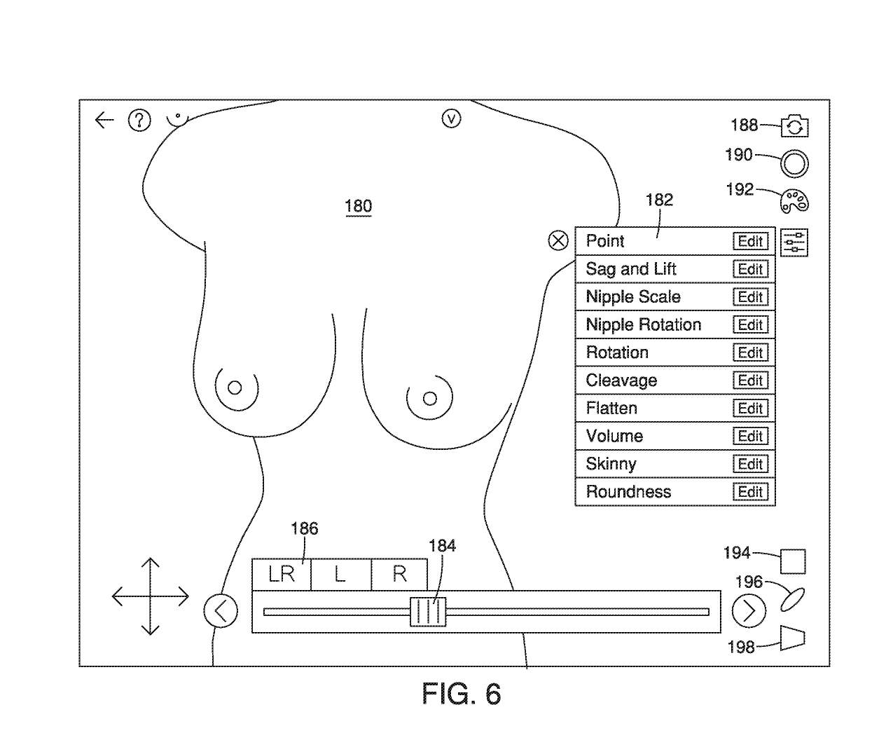

In one implementation, the present invention is implemented as an iOS or Android application that is executed on a mobile device 10 such as a tablet or phone (see FIGS. 6-8). Using high pixel cameras generally present on such tablets, a defined and highly optimized target is tracked and placed (in this case, tracking marker 160 that covers the patient's breasts), and an augmented reality image of proposed breast augmentation is overlaid on the marker such that the patient is able to see her real body with virtual breasts at the location of her real breasts. By use of low polygon graphics and various deformations easily applied by a slider on the user interface, the virtual breasts can be deformed in real time to produce lifelike outcomes of various surgical options.

While the present invention is described primarily in the context of breast augmentation or breast implant surgery, it may be applied to numerous other cosmetic surgical procedures. For instance, the system of the present invention has utility in tummy tuck, liposuction, nose reshaping, facelifts and other cosmetic surgical procedures. In addition, the augmented reality imaging system of the present invention may be applied in other industries having a need for a real time three-dimensional imaging system.

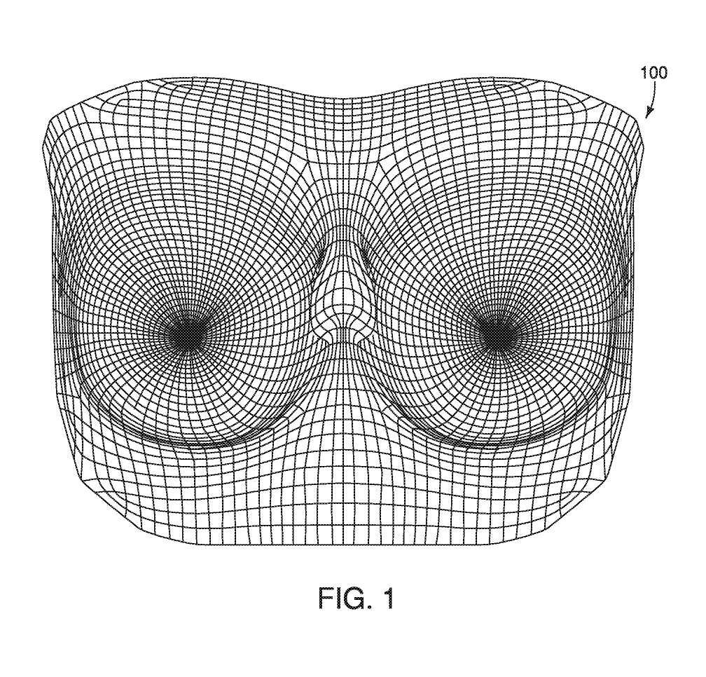

A virtual breast image according to the present invention is created as follows. First, an initial breast mesh 100 is generated from blend shapes that are created using three-dimensional animation, modeling, simulation and rendering software such as, for example. Maya® by Autodesk. A blend shape deforms geometry to create a specific look in a mesh form. In particular, as shown in FIG. 1, initial breast mesh 100 is generated and given an appropriate UV layout or mapping (a two-dimensional image representation of a three-dimensional model's surface).

A system of planar mappings is used to properly deform breast mesh 100 into a realistic form. Planar projections are a subset of three-dimensional graphical projections constructed by linearly mapping points in three-dimensional space to points on a two-dimensional projection plane. A projected point on the plane is chosen such that it is collinear with the corresponding three-dimensional point and the center of projection. The lines connecting these points are referred to as projectors. The center of projection may be thought of as the location of an observer, while the plane of projection is the surface on which the two dimensional projected image of the scene is recorded or from which it is viewed (e.g., photographic negative, photographic print, computer monitor). When the center of projection is at a finite distance from the projection plane, a perspective projection is obtained.

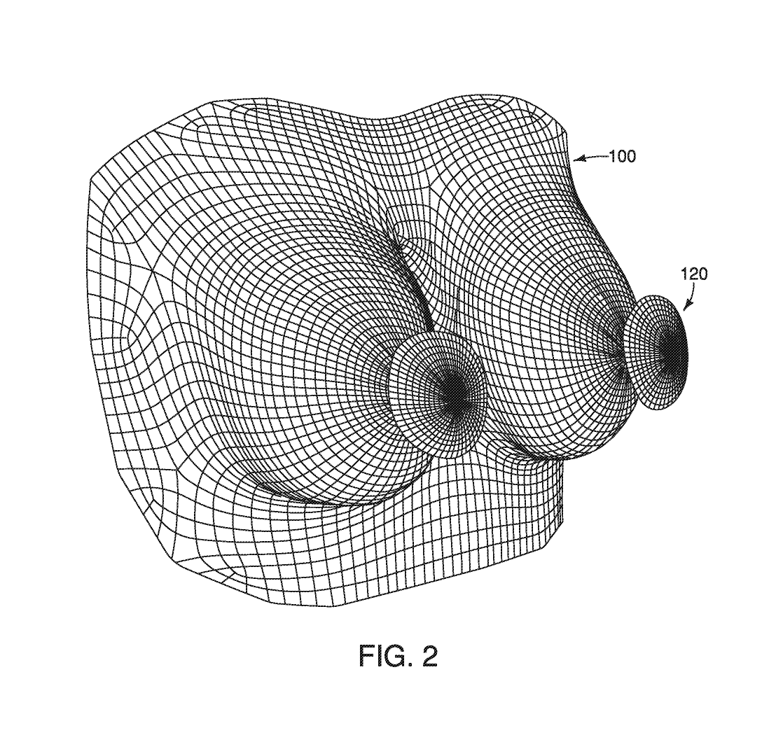

Next, using the existing geometry and the same process as for breast mesh 100, a nipple mesh 120 is generated and given an appropriate UV layout (FIG. 2). The texturing is painted first with a very small nipple texture. That texture is transparent and blends seamlessly with the geometry when applied on top of it using an unlit material. Nipple mesh 120 is given a small offset on each axis so as not to interfere with the previous breast geometry.

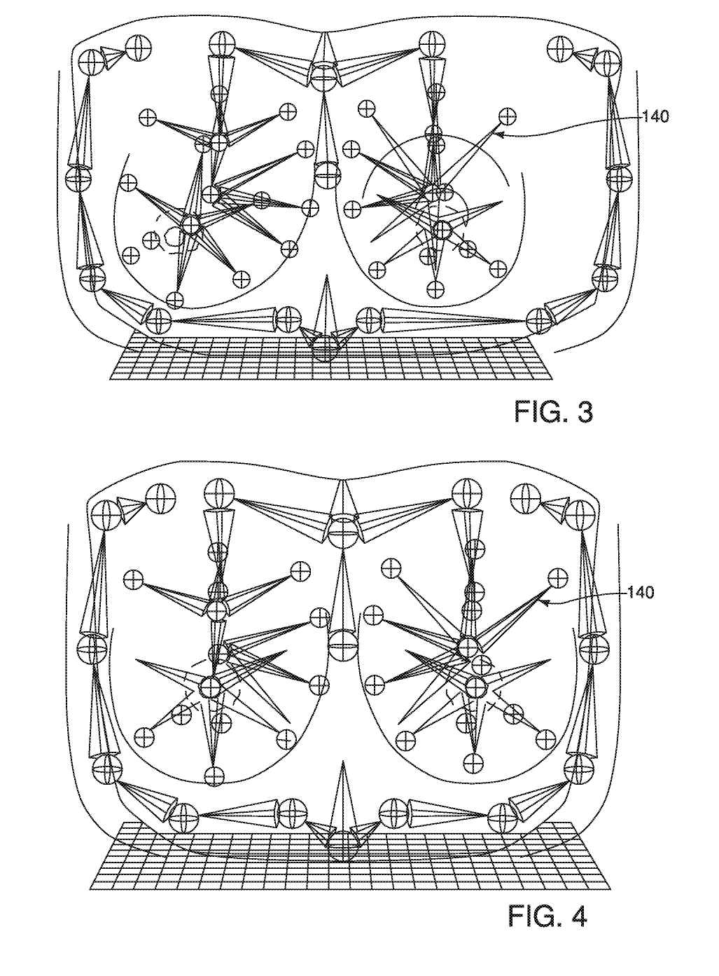

As illustrated in FIGS. 3 and 4, once breast mesh 100 and nipple mesh 120 have been generated and UV maps generated for both meshes, a base joint and curve rig 140 is built in order to generate a number (for instance, twelve) of target breast deformations. In order to properly mask the skin or texture to the underlying model, the base joint and curve rig is applied to obtain flexibility of the model. Rig 140 is a low poly smooth skinned mesh on which weights are painted to make quick targets. Rigging is the process of taking a static mesh, creating an internal digital skeleton, creating a relationship between the mesh and the skeleton (known as skinning, enveloping or binding) and adding a set of controls (sliders, as will be described later) that an end user can use to push and pull the model so that it reflects actual deformations or variations in human breast forms. By using a method of low polygon modeling and vertices, the digital model may be proportionately deformed.

First, an initial blend shape of a right side breast is generated. Then, using a technique of copying and scaling over the X axis, a wrap is deformed and applied in conjunction with the initial blend shape to generate the left side. This does not alter the vertices original placement but copies that deformation to the left side. When both blend shape deformations are triggered, they work in conjunction yet can also be manipulated on their own. Thus, a surgeon can take into account different breast shapes and sizes. A surgeon can deform the left and right breasts individually, or may use a default combination of the left and right breasts.

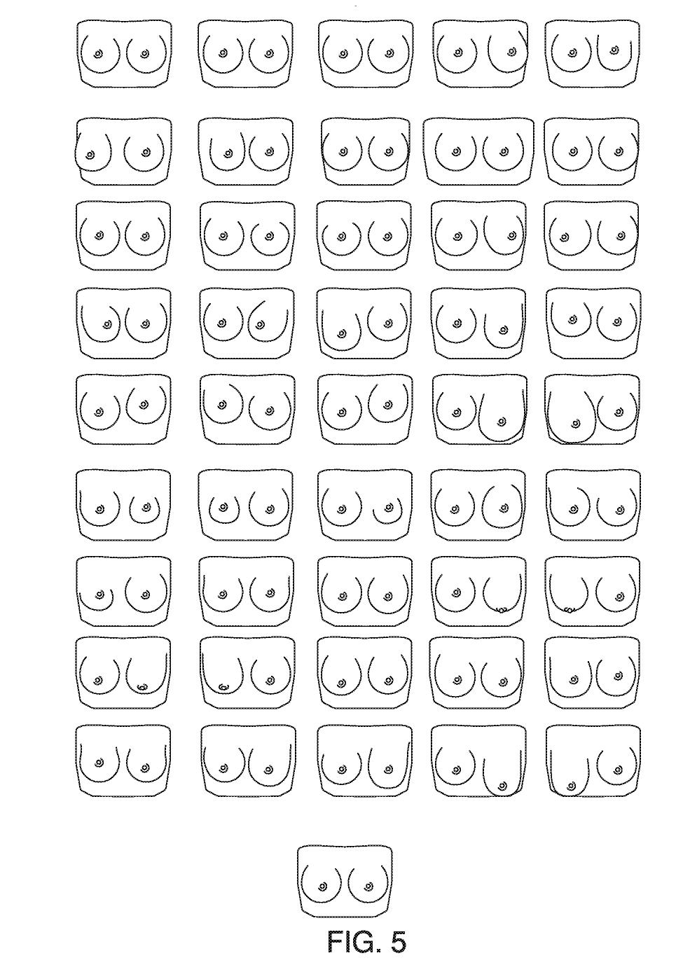

Using vertex manipulation via base joint and curve rig 140, a number of target breast deformations are created. By following the same deformation procedure, the texture of the nipple can also be changed to supply a different shape to the nipple as well as movement up and down on the geometry. In one implementation, ten target breast deformations 182 are created including “point”, “sag and lift”, “nipple scale”, “nipple rotation”, “rotation”, “cleavage”, “flatten”, “volume”, “skinny”, and “roundness” (FIG. 6). As will be described with reference to FIG. 6, target breast deformations 182 may be applied to a slider 184 in user interface 180, which is displayed on mobile device 10, in order to allow the surgeon to push and pull the model to reflect actual deformations in the breast form. FIG. 5 illustrates exemplary breast deformations that are possible from target breast deformations 182.

Next, a base alpha texture is built for blurred edges on the augmented reality mesh, and a number of texture maps are generated to match different skin types. Using the previous LA's allocated for each mesh, the target deformations are painted to have a realistic texture. In one implementation, the target deformations are painted using digital painting and sculpting software such as Mudbox® by Autodesk. A number (such as eight, for instance) of different skin tones from dark to light may be applied to represent slight skin inconsistencies and to generate a realistic texture map. In addition, virtual clothing such as a tank top and bikini may be generated and modeled to fit the various breast deformations.

With all of the target breast deformations applied to the model, the model is imported into a game engine such as Unity® by Unity Technologies and compiled. Inside of Unity, the model is applied to a scene using a camera or mobile vision platform such as, for example, Vuforia® by Qualcomm. By placing the model on an optimized tracking image, an appropriate understanding of where the model will be tracked can be obtained. Deformations 182 may then be manipulated by slider 184 in user interface 180 of mobile device 10 to give the surgeon flexibility to change or adjust any of target modifications 182 on the fly. As shown in FIG. 6, for example, deformations 182 including “point”, “sag and lift”, “nipple scale”, “nipple rotation”, “rotation”, “cleavage”. “flatten”, “volume”, ““skinny”, and “roundness” may be displayed and selectable in user interface 180, such that the selected deformation can be changed or manipulated by use of slider 184. As can be seen in FIG. 6, slider 184 also includes breast selection buttons 186 to select whether the changes made by slider 184 should be applied to both breasts (“LR”), only the left breast (“L”), or only the right breast (“R”).

User interface 180 may include other icons and controls to assist in creating the breast model. For example, user interface 180 may include a camera icon 188 to change the camera from rear facing to forward facing, circle icon 190 to place the mobile device display into a picture capturing mode such that a snapshot is captured by pressing circle icon 190, and palette icon 192 to change the color of the skin and shade of the nipple. In addition, user interface may include zoom icon 194, tilt icon 196 and depth icon 198 for controlling the zoom, tilt and depth of the model.

Next, textures are generated. A base material shader is made using an unlit material. By using unlit materials, more realistic lighting is generated and mobile computer usage is saved. A shadow map is generated using three-dimensional animation software such as Maya® through a process of high dynamic range imaging (HDRI) and global illumination to provide a black and white shadow texture. After assembling the eight textures this shadow mask is applied as a multiply layer. A nipple shader is then built using a system of numbers, and the appropriate size is selected inside of the 0-1 texture file.

Using software such as Unity®, the model is coded to be placed on the fly on a tracking marker 160 that covers the patient's breasts. In one implementation, as shown in FIG. 7A, tracking marker 160 is a printed marker that is attached around the patient's breasts via an elastic band attached to the marker. Tracking marker 160 can be positioned by the surgeon as appropriate considering the body type of the patient. In addition, as marker 160 covers the patient's breasts, the camera does not capture an image of her full nude body.

The virtual breast model (image) generated as described above is then overlaid onto tracking marker 160 using marker based tracking provided by a mobile vision platform such as, for example, Vuforia®. In particular, as the patient views the Wad or other mobile device 10 with tracking marker 160 coveting her breasts (FIG. 7B), the virtual breast model generated is set onto marker 160, such that she sees the virtual breasts 202 on her real body 200 (FIG. 7C), By selecting a deformation 182 and using slider 184, the surgeon can quickly and easily deform the virtual breasts in various ways while the patient watches, allowing a surgeon to create accurate breast models in less than 60 seconds. Thus, the surgeon can allow the patient to see options for surgeries using different sized and shaped implants, and can also demonstrate how the patient's breasts will deform based on different procedures.

Once the surgeon and patient have decided on a particular breast augmentation, the image of that augmentation (patient's body with virtual breasts overlaid) is stored in the memory of the mobile device. In one implementation, mobile device 10 transmits the stored image to a confidential website, and the patient can log into that site from home and view one or more proposed augmentations from their own desktop or mobile device, and if desired share that information with their spouse or significant other.

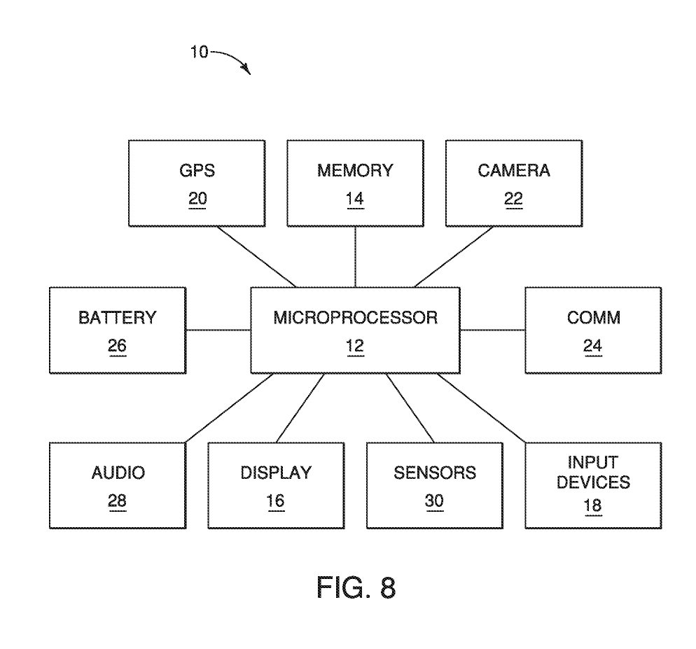

In one embodiment, the present invention is implemented as a mobile application or program stored in a memory and executed by a microprocessor on a tablet computer, smart phone or other mobile device, such as mobile device 10 of FIG. 8. In one implementation, mobile device 10 is an iPad® by Apple. As illustrated in FIG. 8, mobile device 10 may include, without limitation, a microprocessor or central processing unit (CPU) 12 and memory 14. Memory 14 may be any non-transitory computer-readable storage medium such as, without limitation, RAM (random access memory), DRAM (dynamic RAM), ROM (read only memory), magnetic and/or optical disks, etc. Memory 14 may be configured and partitioned in various known fashions. Generally speaking, memory 14 typically includes a static component (such as ROM) where the operating system (iOS or Android, for example) and system files are stored, as well as additional storage for mobile applications (“apps”) that are executed by microprocessor 12, image files, and other data, utilities, etc. Memory 14 also typically includes a non-static and faster access portion (such as DRAM) where critical files that need to be quickly accessed by microprocessor 12 (such as operating system components, application data, graphics, etc.) are temporarily stored.

Mobile device 10 also includes a display 16, preferably a touch screen, and may also include additional user input devices 18 such as buttons, keys, etc. Mobile device 10 may include a GPS (global positioning system) unit 20 and camera 22. Mobile device 10 further includes communication components 24 that permit device 10 to exchange communications and data with other devices, establish Internet, Wi-Fi and Bluetooth connections, and so on, including the exchange of data and images with a server. Power is supplied to mobile device 10 via battery 26. Device 10 also includes audio output or speaker 28, and may also include sensors 30 such as motion detectors, accelerometers, gyroscopes, etc.

Mobile device 10 is merely one exemplary framework of a computing environment in which the present invention may be implemented, and may include different, additional or fewer components and functionality than the mobile device 10 that is illustrated in FIG. 8. The present invention may be implemented in any suitable computing environment including smart phones, tablet computers, digital imaging equipment, personal computers and the like.

1. An augmented reality imaging system for cosmetic surgical procedures comprising:
generating a virtual and deformable image of a body part of a patient that is to be subject to a cosmetic surgical procedure;
covering the body part with a tracking marker;
generating an image of the patient with the tracking marker covering the body part;
overlaying the virtual and deformable image on the body part; and
displaying the image to the patient.

2. The augmented reality imaging system of claim 1, wherein the body part is a breast.

3. The augmented reality imaging system of claim 2, wherein the image is displayed on a tablet.

4. The augmented reality imaging system of claim 3, wherein a display on the tablet includes sliders that can be moved in order to deform the virtual image of the body part.

5. The augmented reality imaging system of claim 2, wherein the step of generating the virtual and deformable image of the breast comprises:
generating a breast model from an initial breast mesh and an initial nipple mesh;
generating a plurality of target breast deformations by deforming the breast model using low polygon modeling and vertices; and
applying the target breast deformations to the breast model.
